(19) United States
(12) Patent Application Publication
(10) Pub. No.: US 2022/0017453 A2
(43) Pub. Date: Jan. 20, 2022

(54) IGM COMPOSITIONS AND METHODS OF MUCOSAL DELIVERY OF THESE COMPOSITIONS

(71) Applicant: University of Louisiana at Lafayette, Lafayette, LA (US)

(72) Inventors: Ruth Ruprecht, San Antonio, TX (US); Siqi Gong, San Antonio, TX (US)

(21) Appl. No.: 17/052,671

(22) PCT Filed: May 6, 2019
(86) PCT No.: PCT/US2019/000022

§ 371 Date: Nov. 3, 2020

Related U.S. Application Data
(60) Provisional application No. 62/667,213, filed on May 4, 2018.

(51) Int. Cl. C07C 209/68; C07F 1/00; C07C 227/32; B01J 31/18

(52) U.S. Cl. CPC C07C 209/68; C07F 1/005; C07C 211/48; B01J 31/1805; C07C 227/32

(57) ABSTRACT

Described herein are methods of inducing an immune response directed towards preventing or reducing the risk of a human immunodeficiency virus (HIV) infection in a mammalian subject. The subject is administered an effective amount of a composition containing IgM antibodies directed to an epitope of an envelope protein of the HIV virus. Also disclosed here are vaccine compositions comprising IgM antibodies directed to one or more epitopes of one or more human immunodeficiency virus envelope proteins. Also disclosed are recombinant immunoglobulin M compositions containing a Fcγ fragment of an immunoglobulin G.

### GOVERNMENT SUPPORT

This invention was made with government support under Grant Nos. R01 AI100703 and P01 AI048240 awarded by the National Institutes of Health. The government has certain rights in the invention.

### FIELD OF INVENTION

This disclosure relates to immunoglobulin M (IgM) compositions that are administered to patients to prevent, manage, or treat infectious diseases. This disclosure also relates to compositions and methods of inducing an immune response directed towards preventing or reducing the risk of HIV infection in a mammalian subject.

### BACKGROUND

Worldwide, about 90% of all human immunodeficiency virus (HIV) infections occur through mucosal exposure and almost always involve CCR5 (R5)-tropic HIV strains that are relatively difficult to neutralize (tier 2). After acute infections, IgM is the first antibody (Ab) class to respond. IgM is the only Ab present in all vertebrates. It is required for the maturation of immunoglobulin G (IgG) responses, regulation of B cell development, modulating inflammatory responses, agglutination of pathogens, and clearance of apoptotic cells via complement activation. IgM exists as dimer on the surface of B cells, forming the B-cell receptor. Plasma IgM is mainly pentameric and contains the joining (J)-chain. At mucosal sites, IgM is produced locally by plasma cells in the lamina propria. After its production, IgM binds to the polymeric immunoglobulin receptor (pIgR) expressed on the basolateral surface of the epithelial barrier to form pIgR-IgM complexes. The latter are transported across the epithelial monolayer in transcytotic vesicles and released at the luminal side through proteolytic cleavage of pIgR. This process results in the release of secretory component (SC) that remains associated with IgM, thus generating secretory IgM (SIgM). The role of IgM in preventing HIV transmission is currently unknown. Induction of protective IgM responses has not been a defined goal for vaccine development due to the waning of early IgM responses that are replaced by IgG, IgA, and other Ig class responses that arise by programmed class switching. Thus, IgM is not usually considered to play an important role in long-term immunity.

### SUMMARY

Disclosed herein are compounds and methods addressing the shortcomings of the art, and may provide any number of additional or alternative advantages, including more effective compositions and methods for prevention, management, or treatment of infectious diseases and vaccine development.

Embodiments of the methods of prevention, management, or treatment of an infectious disease in a mammalian subject include the administration of IgM compositions. The IgM compositions disclosed here induce an immune response directed towards preventing or reducing the risk of infection in a mammalian subject. In an embodiment, the infection is a HIV infection. An example of a method includes administering to the mammalian subject an effective amount of a composition containing IgM antibodies directed to an epitope of an envelope protein of the HIV virus. The envelope protein of the HIV virus can be HIV-1 gp120 polypeptide. The composition containing IgM antibodies can be formulated for a mucosal administration. Another example of a method of prevention, management, or treatment of an infectious disease includes administering a composition containing IgM antibodies directed to an epitope of an envelope protein of the HIV virus to a mucosal layer in a mammalian subject. The mucosal layer can be a rectal mucosal layer.

Disclosed here are IgM-containing compositions and methods of including IgM in the form of a microbicide and inducing an immune response directed towards preventing or reducing the risk of HIV infection in a mammalian subject. Embodiments include vaccine compositions comprising IgM antibodies directed to an epitope of a HIV-1 gp120 envelope protein of the HIV virus. Embodiments include vaccine compositions comprising an epitope of a HIV-1 gp120 envelope protein of the HIV virus and also IgM antibodies directed to the epitope of a HIV-1 gp120 envelope protein. Certain embodiments include immune complexes with IgM antibodies and either the recombinant HIV Env protein or subunits thereof. Certain embodiments include immune complexes with IgM antibodies and recombinant bacteriophages that contain one or more mimotopes of HIV Env (or other HIV proteins) that have been linked to vaccine-induced protection. Certain embodiments include immune complexes with IgM antibodies and recombinant bacteriophages, which contain HIV Env mimotopes linked to neutralization of tier 2 HIV or simian-human immunodeficiency virus (SHIV) strains.

Embodiments of recombinant immunoglobulin M compositions include a Fcγ fragment of an immunoglobulin G connected to a carboxy terminus of a joining chain of an immunoglobulin M. In an embodiment, the Fcγ fragment of the immunoglobulin G is connected to the carboxy terminus of the joining chain of the immunoglobulin M via a linker. The linker can be a glycine- and serine-rich linker. Embodiments of recombinant immunoglobulin M compositions include a Fcγ fragment of an immunoglobulin G connected to a carboxy terminus of a constant region of a light chain of immunoglobulin M. In an embodiment, the Fcγ fragment of the immunoglobulin G is connected to the carboxy terminus of the constant region of the light chain of the immunoglobulin M via a linker. The linker can be a glycine- and serine-rich linker.

Embodiments of recombinant immunoglobulin M compositions include five monomers of a bispecific antibody containing: a first constant region linked to a first variable region, wherein the first variable region includes a variable region of a first heavy chain and a variable region of a first light chain capable of specifically binding to a first epitope of a pathogen; and a second constant region linked to a second variable region, wherein the second variable region includes a variable region of a second heavy chain and a variable region of a second light chain capable of specifically binding to a second epitope of the pathogen. The recombinant immunoglobulin M composition can further include a joining chain connecting the first constant region of a heavy chain of a first monomer to a third constant region of a heavy chain of a second adjacent monomer to form a pentameric immunoglobulin M composition. The first epitope and the second epitopes can be two different epitopes of one human immunodeficiency virus envelope protein. For example, this human immunodeficiency virus envelope protein can be gp120. In an embodiment, the first epitope can be from a first human immunodeficiency virus envelope protein and the second epitope can be from a second human immunodeficiency virus envelope protein. As an example, the first epitope can be a carbohydrate dependent epitope related to the V3 loop of the human immunodeficiency virus envelope protein gp120. The second epitope can be a part of a V2 loop of the human immunodeficiency virus envelope protein gp120. The second epitope can be a part of a CD4 binding site of the human immunodeficiency virus envelope protein gp120. In an embodiment, the recombinant immunoglobulin M composition contains the variable region of the first light chain is connected to the variable region of the first heavy chain, which is connected to the first constant region, and the variable region of the second light chain is connected to the variable region of the second heavy chain, which is connected to the second constant region. The first constant region is linked to the variable region of the first heavy chain via a first glycine- and serine-rich linker. And, similarly, the second constant region is linked to the variable region of the second heavy chain via a second glycine- and serine-rich linker.

Numerous other aspects, features and benefits of the present disclosure may be made apparent from the following detailed description taken together with the drawings. Pharmaceutical compositions can include the IgM compositions described herein along with other components, or ingredients depending on desired prevention and treatment goals. It should be further understood that both the foregoing general description and the following detailed description are exemplary and explanatory and are intended to provide further explanation of the inventions as claimed.

### BRIEF DESCRIPTION OF THE DRAWINGS

The present disclosure can be better understood by referring to the following figures. The emphasis is instead placed upon illustrating the principles of the disclosure.

FIG. 1A is a photographic image of a sodium dodecyl sulfate polyacrylamide gel electrophoresis (SDS-PAGE) analysis for expression of 33C6-IgM (recombinant polymeric monoclonal IgM was generated from the neutralizing monoclonal IgG1 antibody 33C6-IgG1) and 33C6-IgG1.

FIG. 1B and FIG. 1C are graphical representations of the results from a dynamic light scattering assay to determine particle size of 33C6-IgM and 33C6-IgG1, respectively. Data are representative of 4 independent experiments.

FIG. 1D is a graphical representation of the 33C6-IgM binding to consensus HIV clade C peptides of V3 and Env proteins.

FIG. 1E is a graphical representation of the binding avidity of 33C6-IgM and 33C6-IgG1. Data are representative of two independent experiments.

FIG. 1F is a graphical representation of the binding affinities of captured 33C6-IgM for solution phase SHIV-1157ip gp120, with representative concentration series of Surface Plasmon Resonance (SPR) sensorgrams ranging from 41 picomolar (pM) to 10 nanomolar (nM) of gp120. Global fits to a 1:1 binding model are overlaid in black. Average association rate constant (kₐ), dissociation rate constant (k_{d}), and equilibrium dissociation binding constant (K_{D}) with standard errors from 3 replicates are shown in the insert.

FIG. 1G is a graphical representation of the binding affinities of captured 33C6-IgG1 for solution phase SHIV-1157ip gp120, with representative concentration series of SPR sensorgrams ranging from 41 picomolar (pM) to 10 nanomolar (nM) of gp120. Global fits to a 1:1 binding model are overlaid in black. Average kₐ, k_{d}, and K_{D} with standard errors from 3 replicates are shown in the insert.

FIGS. 2A and 2B are graphical representations of the neutralization assay and virion capture assay by 33C6 mAbs. FIG. 2A is the graphical representation of the neutralization of the challenge virus, SHIV-1157ipEL-p, by 33C6-IgM and 33C6-IgG1. FIG. 2B is the graphical representation of the capture of physical virus (pVirus) particles by 33C6-IgM and 33C6-IgG1. Data are representative of two independent experiments. Error bars, mean ±SEM. VRC01-IgG1 was used as positive control, while Fm-6-IgG1 and IgM isotype control were used as negative controls.

FIGS. 3A-3C are diagrammatic representations of the experimental design of infection of the Rhesus macaques. The animals were randomized into three groups (n=6 per group). FIG. 3A shows the timeline of the study, where Group 1 received 33C6-IgM intrarectally (i.r.) (red arrow). FIG. 3B shows the timeline of the study, where Group 2 was given 33C6-IgG1 i.r. (blue arrow). FIG. 3C shows the timeline of the study, where Group 3 received only phosphate-buffered saline (PBS) i.r. (empty arrow). One of either 33C6-IgM, 33C6-IgG1, or PBS was given 30 min before the virus challenge. In each of FIGS. 3A-3C, the black arrow indicates the time when a high-dose i.r. SHIV-1157ipEL-p challenge with 31.5 50% animal infectious doses (31.5 AID₅₀) was given to all three groups.

FIGS. 4A-4D are graphical representations of the plasma viral loads in macaques challenged with SHIV-1157ipEL-p. Plasma viral RNA is expressed as a log scale of the viral RNA copies per milliliters (log vRNA copies/ml). FIG. 4A is a graphical representation of the plasma viral loads in macaques in Group 1, who received 33C6-IgM. FIG. 4B is a graphical representation of the plasma viral loads in macaques in Group 2, who received 33C6-IgG1. FIG. 4C is a graphical representation of the plasma viral loads in macaques in Group 3 (controls). In each of FIGS. 4A-4C, the black dotted line indicates the lower limit of viral RNA detection (50 copies/ml). FIG. 4D is a graphical representation of the Kaplan-Meier analysis of time until peak viremia for all three Groups. Log-rank test was used to determine significance.

FIGS. 5A-5C are graphical representations of the titers of anti-SHIV plasma antibodies and their neutralization profiles against the challenge virus, SHIV-1157ipEL-p. FIG. 5A shows the titers of gp120-binding antibodies in plasma from rhesus macaques (RMs) with breakthrough infection. FIG. 5B shows the in vitro neutralization of SHIV-1157ipEL-p at day 42 post challenge and FIG. 5C shows the in vitro neutralization of SHIV-1157ipEL-p at day 82 post challenge. Data are representative of two independent experiments; each sample was analyzed in duplicate.

FIGS. 6A-6D are illustrations of the different strategies employed to generate recombinant clones containing Fcγ and IgM. In FIG. 6A, the human IgG constant heavy chain CH2-CH3 regions were fused via three repeats of the tetra-glycine-serine peptide linker (G₄S) to the C-terminus of the light chain constant region. This strategy allows ten copies of Fcγ to be incorporated into every pentameric rIgM, as shown in FIG. 6C. In FIG. 6B, the CH2-CH3 region of the human IgG heavy chain constant region was connected to the C-terminus of the Joining (J) chain through the same (G₄S) 3 linker described in FIG. 6A. This design is expected to yield only one Fcγ per pentameric IgM molecule given that pentameric IgM contains just one J chain, as shown in FIG. 6D.

FIG. 7 is a photographic image of a SDS-PAGE and western blot analysis for the presence of Fcγ in pentameric IgM compositions corresponding to FIGS. 6C and 6D.

FIG. 8A is a schematic presentation of bispecific IgM heavy and light chain. The direction of VH and VL can be switched within each scFv. The position of scFv 1 and scFv 2 are interchangeable as well. FIG. 8B is a photographic image of a SDS-PAGE gel probed with anti-human μ chain antibody for the presence of a total of eight combinations of heavy and light chain plasmids for a bispecific IgM, termed PGT121-VRC01-IgM.

FIGS. 9A and 9B are graphical representations of the competitive binding assays to verify epitope specificity of a bispecific IgM, PGT121-VRC01-IgM using two mAbs: PGT121-IgG1 and VRC01-IgG1, respectively.

### DETAILED DESCRIPTION

Reference will now be made to the exemplary embodiments illustrated in the drawings, and specific language will be used here to describe the same. It will nevertheless be understood that no limitation of the scope of the invention is thereby intended. Alterations and further modifications of the inventive features illustrated here, and additional applications of the principles of the inventions as illustrated here, which would occur to one skilled in the relevant art and having possession of this disclosure, are to be considered within the scope of the invention.

Embodiments include compositions containing IgM antibodies directed to one or more epitopes of one or more surface proteins of a pathogen. Embodiments include compositions containing IgM antibodies directed to one or more episodes of one or more bacterial surface proteins. Embodiments include compositions are containing IgM antibodies directed to one or more epitopes of surface proteins of one or more parasite or fungus pathogen. Embodiments include compositions containing IgM antibodies directed to one or more viral surface proteins; for enveloped viruses, these compositions containing IgM antibodies can be directed to the viral envelope proteins. The compositions containing IgM antibodies can be directed to an epitope of an envelope protein of the HIV virus. The methods of inducing an immune response directed towards preventing or reducing the risk of HIV infection in a mammalian host include administering to a mammalian subject an effective amount of a composition containing IgM antibodies directed to an epitope of an envelope protein of the HIV virus. The methods of preventing HIV infection include a mucosal administration of a microbicide containing IgM antibodies directed to an epitope of an envelope protein of the HIV virus. An epitope is a set of amino acids, either contiguous or not, by themselves or as part of a larger sequence, that bind to an antibody generated in response to such sequence. Epitopes can be sequences identical to the native sequence of a surface protein of a pathogen, as well as modifications to the native sequence, such as deletions, additions and substitutions (generally conservative in nature). When an antibody is directed to an epitope, such antibody is capable of recognizing and binding to that epitope.

Preclinical vaccine efficacy studies rely on nonhuman primate models, especially Indian-origin rhesus macaques (RMs). However, the envelope of the simian immunodeficiency virus (SIV) is so divergent that antibodies against HIV do not recognize the SIV envelope proteins. Therefore, simian-human immunodeficiency viruses (SHIVs) have been constructed. These chimeras carry HIV env in a SIVmac239 backbone. The R5 SHIV/RM model was used to assess the protective potential of recombinant human anti-HIV Env monoclonal antibodies (mAbs) by passive immunization.

In an embodiment, the IgM antibodies directed to an epitope of an envelope protein of the SHIV virus is the anti-HIV antibody—33C6-IgM. The majority of the passively immunized RMs was completely protected. In IgM-treated RMs with breakthrough infection, there was a trend for earlier development of autologous neutralizing Abs than in virus-only controls. The 33C6-IgM antibodies neutralized SHIV, captured physical virus particles, and depleted infectious virions in vitro, suggesting potential protective mechanisms. IgM can be an effective first-line of defense at mucosal barrier. In certain embodiments, the IgM compositions can be used as topical microbicides. In an embodiment, the recombinant IgM compositions are administered as a gel to prevent HIV infections.

The term “effective amount” as used herein, means that amount of IgM composition that elicits the biological or medicinal response in a tissue system, animal or human that is being sought by a researcher, veterinarian, medical doctor or other clinician, which includes a reduction in symptoms or a reduction in the infectious agent load in the biological system.

The 33C6-IgM antibodies target a protruding element of HIV Env, the conserved V3 loop crown that is easily accessible in the challenge virus. Consequently, the 33C6-IgM antibodies potently neutralized the challenge SHIV and not only captured most of the physical virions present in the stock, but also removed close to 100% of the infectious particles. The IgG1 isotype was equally effective in eliminating infectious virions by capture. These characteristics can be explained by the binding profiles for 33C6-IgM and 33C6-IgG1 as assessed by avidity indices and SPR analyses; by both measures, 33C6-IgM showed tighter binding. The affinity of 33C6-IgM for the soluble challenge virus gp120 was extraordinarily high with a K_{D} in the low picomolar range indicating that binding was 52-fold tighter than binding of the IgG1 isoform.

The dual action—direct neutralization and efficient infectious virion capture—is the underlying basis for the protection that was observed in vivo for both mAb forms of 33C6. The pentameric IgM can efficiently trap incoming virus by crosslinking and prevent mucosal transmission through immune exclusion. Intra-luminal administration of mAbs of different Ig classes appears to prevent SHIV transmission; this includes recombinant monoclonal IgM, IgG, and dimeric IgA (dIgA). Other groups have focused on vaginal administration of anti-HIV IgG1 neutralizing mAbs. The passive mucosal immunizations with human dIgA1 and dIgA2 isotypes involved mAbs with almost identical epitope specificity as that of the 33C6-IgM antibodies, namely HGN194. Both series of mAbs target the conserved gp120 V3 loop crown in R5-tropic HIV strains. HGN194 was isolated as an IgG1 from an infected person harboring an HIV clade AG circulating recombinant form. In contrast, 33C6-IgG1 was initially cloned from a single memory B cell from a RM with chronic clade C SHIV infection. The mAb 33C6-IgG1 specifically recognized a conformational mimotope representing the V3 loop.

Anti-HIV neutralizing mAbs have been class-switched from IgG to IgM, such as the IgG1 mAbs 2F5, 4E10, and 2G12. The mAbs 2F5 and 4E10 target epitopes in the membrane proximal external region (MPER) of HIV gp41 protein. Class switching from IgG1 to IgM resulted in loss of neutralizing activity. In contrast, the IgM isoform of 2G12, a mannose-dependent neutralizing Ab with epitopes located on the glycan shield, not only retained the ability to neutralize HIV but actually neutralized the virus up to 28-fold-more efficiently in PBMC cultures than the corresponding IgG1 isoform. The contrasting results obtained with anti-MPER and anti-glycan mAbs can be explained by differential epitope accessibility. The 33C6 antibodies used herein target the readily accessible, conserved V3 loop crown epitope of gp120. This translated into efficient neutralization and virion capture by IgM and protection of RMs against a mucosal SHIV challenge.

IgM is the first antibody class to respond to infection or immunization. IgM responses have been used to diagnose new infections by various pathogens. The very first free plasma antibodies detected in acute HIV infection involved IgM; these antibodies had autologous anti-gp41 specificity. They appeared as early as five days after plasma viremia became detectable and were also found as virion-IgM complexes. Mathematical modeling showed that the early anti-gp41 IgM responses did not affect plasma viremia and thus were not felt to benefit the host. This important study focused on virus-specific IgM responses after the HIV transmission had already occurred.

The recombinant IgM compositions can include a plurality of monomers. In an embodiment, the recombinant IgM composition can be pentameric. In an embodiment, the recombinant IgM composition can be hexameric. Embodiments include methods of passive. immunoprophylaxis with anti-HIV IgM compositions before virus exposure. This includes mAbs directed to well-characterized epitopes. Passive immunization data can serve as blue prints for immunogen selection and design. In the passive immunization methods with the 33C6-IgM antibodies, unusually early appearance of SHIV-neutralizing antibody responses were observed in the context of breakthrough infection despite passive IgM immunization. IgM responses can be long-lived and contribute to long-term protection. Recombinant, monoclonal IgM protects against mucosal SHIV acquisition.

Recombinant IgM against HIV Env (as 33C6-IgM) accelerate the generation of neutralizing antibodies in the rhesus monkeys, as shown by example in this disclosure. The intrarectal passive immunization with anti-HIV Env IgM had breakthrough infection after intrarectal SHIV challenge. The IgM-SHIV immune complexes were probably formed in the mucosal cavity and taken up by cells of the immune system; thus, stimulating the early appearance of neutralizing antibodies. In an embodiment, the recombinant IgM antibodies were delivered to induce protective antibodies against HIV.

In certain embodiments, recombinant IgM antibodies are constructed to bind specifically to an epitope of the HIV Env protein. For example, the immunoglobulin (Ig) heavy and light chain genes are cloned into an appropriate Ig backbone vector. This nucleic acid composition is then transfected into cells for production of the IgM antibodies. These antibodies are then purified and utilized to generate immune complexes with IgM. These immune complexes contain the IgM antibodies and either the recombinant HIV Env protein or subunits thereof

In certain embodiments, recombinant IgM antibodies are constructed to bind specifically to bacteriophage proteins. This recombinant anti-phage IgM antibodies are purified and used in the production of immune complexes with recombinant bacteriophages that contain one or more mimotopes of HIV Env (or other HIV proteins) that have been linked to vaccine-induced protection. In certain embodiments, the recombinant IgM antibodies are used in the production of immune complexes with recombinant bacteriophages, which contain HIV Env mimotopes linked to neutralization of tier 2 HIV or SHIV strains. These mimotopes are the “internal images” of the paratopes of antibodies that neutralize tier 2 strains. These mimotopes are not recognized by antibodies that only neutralize tier 1 HIV or SHIV strains. Tier 2 viruses are difficult to neutralize, and neutralizing antibodies frequently only inhibit laboratory-adapted tier 1 viruses or clinical tier 1 virus isolates that are unusually sensitive to neutralization. In certain embodiments, the recombinant IgM antibodies as part of immune complexes can be utilized to induce protective antibodies against other viruses or pathogens.

Certain embodiments of the disclosure include recombinant IgM compositions containing the Fcγ fragment of the IgG. These recombinant IgM compositions expand the effector function of IgM compositions. These compositions interact strongly with the complement system. In order to provide lgM with the ability to mediate antibody dependent cellular cytotoxicity (ADCC) with natural killer (NK) cells destroying targeted cells, the Fcγ region from IgG were cloned into various positions on the pentameric IgM. These compositions also extended the half-lives of the recombinant IgMs. The recombinant IgM has a high avidity and strong complement activation. In an embodiment, the Fcγ fragment from human IgG1 was cloned into the C terminus of the light chain constant region. In an embodiment, the Fcγ fragment from human IgG1 was cloned into the C terminus of the J chain. These recombinant compositions have a strong ability to activate complement while at the same time recruiting NK cells to provide a second mechanism of cell killing. This is a safeguard against mechanisms that pathogens may have to circumvent complement-mediated defenses. The half-life of these recombinant IgMs are extended over natural lgMs that have half-lives in the order of maximally one week, as the recombinant IgMs enable recycling through the neonatal Fc receptor (FcRn).

In an embodiment of a method of making a recombinant IgM composition, the human IgG constant heavy chain CH2-CH3 regions were fused via three repeats of the tetra-glycine-serine peptide linker (G₄S)₃ to the C-terminus of the light chain constant region. The first 112 amino acids of the human IgG1 heavy chain constant were excluded to remove all of the cysteines associated with inter-chain disulfide bond formation in order to prevent Fcγ dimerization with other chains. This embodiment allows ten copies of Fcγ to be incorporated into every pentameric rIgM. For example, to express this IgM-Fcγ composition, Expi293 cells were co-transfected with three different plasmids encoding (i) the IgM heavy chain, (ii) the J chain, and (iii) a recombinant altered light chain containing the CH2-CH3 region of the human IgG heavy chain constant region fused to the C-terminus of the light chain constant region via the (G₄S)₃ linker.

In an embodiment of a method of making a recombinant IgM composition, the CH2-CH3 region of the human IgG heavy chain constant region was cloned to the C-terminus of the joining (J) chain through the (G₄S)₃ linker described above. This recombinant composition contains only one Fcγ per pentameric IgM molecule given that pentameric IgM contains just one J chain. For example, to express this IgM-Fcγ composition, Expi293 cells were co-transfected with three different plasmids encoding (i) an IgM heavy chain expression plasmid, (ii) the corresponding light chain expression plasmid, and (iii) the expression plasmid with the altered J chain—the CH2-CH3 region of the human IgG heavy chain constant region cloned to the C-terminus of the J chain through the (G₄S)₃ linker.

Embodiments of recombinant IgM compositions include several monomers of a bispecific antibody containing: a first constant region linked to a first variable region and a second constant region linked to a second variable region. The first variable region includes a variable region of a first heavy chain and a variable region of a first light chain capable of specifically binding to a first epitope of a pathogen. The second variable region includes a variable region of a second heavy chain and a variable region of a second light chain capable of specifically binding to a second epitope of the pathogen. The recombinant immunoglobulin M composition can further include a joining chain connecting the first constant region of a heavy chain of a first monomer to a third constant region of a heavy chain of a second adjacent monomer to form a pentameric IgM composition. In an embodiment, the recombinant IgM composition can be hexameric and not include a joining chain. The first epitope and the second epitopes can be two different epitopes of a surface protein of a pathogen. The first epitope and the second epitopes can be two different epitopes of two different surface proteins.

In an embodiment, the first epitope and the second epitopes can be two different epitopes of one human immunodeficiency virus envelope protein. For example, this human immunodeficiency virus envelope protein can be gp120. In an embodiment, the first epitope can be from a first human immunodeficiency virus envelope protein and the second epitope can be from a second human immunodeficiency virus envelope protein. As an example, the first epitope can be a carbohydrate dependent epitope related to the V3 loop of the human immunodeficiency virus envelope protein gp120. The second epitope can be a part of a V2 loop of the human immunodeficiency virus envelope protein gp120. The second epitope can be a part of a CD4 binding site of the human immunodeficiency virus envelope protein gp120.

In an embodiment, the recombinant immunoglobulin M composition contains the variable region of the first light chain is connected via a linker to the variable region of the first heavy chain, which is connected to the first constant region via a linker. The variable region of the second light chain is connected via a linker to the variable region of the second heavy chain, which is connected to the second constant region via a linker. In another embodiment, the recombinant immunoglobulin M composition contains the variable region of the first heavy chain is connected via a linker to the variable region of the first light chain, which is connected to the first constant region via a linker. The variable region of the second heavy chain is connected via a linker to the variable region of the second light chain, which is connected to the second constant region via a linker. In another embodiment, the recombinant immunoglobulin M composition contains the variable region of the first heavy chain is connected via a linker to the variable region of the first light chain, which is connected to the first constant region via a linker. The variable region of the second light chain is connected via a linker to the variable region of the second heavy chain, which is connected to the second constant region via a linker. In another embodiment, the recombinant immunoglobulin M composition contains the variable region of the first light chain is connected via a linker to the variable region of the first heavy chain, which is connected to the first constant region via a linker. The variable region of the second heavy chain is connected via a linker to the variable region of the second light chain, which is connected to the second constant region via a linker. Linkers can include flexible linkers like glycine and serine-rich flexible linkers, such as the (G₄S)₃ linker or the GSAGSAAGSGEF linker, or alpha helix-forming rigid linkers, such as (EAAAK)n.The first constant region is linked to the variable region of the first heavy chain via a first glycine- and serine-rich linker. And, similarly, the second constant region is linked to the variable region of the second heavy chain via a second glycine- and serine-rich linker.

Embodiments of recombinant immunoglobulin M compositions include single chain variable fragments (ScFvs) of the antibodies combined with linkers. Linkers can include flexible linkers like glycine and serine-rich flexible linkers, such as the (G₄S)₃ linker or the GSAGSAAGSGEF linker, or alpha helix-forming rigid linkers, such as (EAAAK)n.

The heavy chain backbone was derived from the human IgM, the constant μ chain. Light chain can be either the κ or the λ chain. Combining multiple specificities in one molecule can have several advantages compared to using two or more single mAbs prepared separately. Targeting more than one epitope concurrently will increase potency while decreasing the chance of escape mutants. Having a chance to interact with two or three epitopes at the same time within the context of a pentameric structure is likely to yield very high avidity. Using the pentameric lgM as a platform will provide the high avidity given the multivalency of the IgM molecule. Costs could be significantly lowered by having to generate, characterize and test just one recombinant mAb as opposed to two or three.

Certain embodiments of the disclosure here include multi-specific IgMs. An embodiment incudes bispecific IgMs. In an embodiment, these IgMs were designed and generated as follows. The heavy (VH) and light (VL) chain variable genes of anti-HIV envelope (Env) monoclonal antibodies (mAbs) were cloned. Single-chain variable fragment (scFv) molecules were generated, which utilized 3 repeats of the tetra-glycine-serine peptide linker (G₄S)₃ to connect the VH and VL segments. The scFvs of mAbs in either the VH→VL (scFv_{VHL}) or VL→VH (scFv_{VLH}) direction were linked to the heavy chain constant region (Cμ) or light chain constant region (Cλ or Cκ) through (G₄S)₃ to generate scFv_{VHL/VLH}-Cλs and scFv_{VHL/VLH}-Cλs (or scFv_{VHL/VLH}-Cκs). The plasmid expressing scFv_{VHL/VLH}-Cμ of a given specificity was co-transfected with a plasmid expressing scFv_{VHL/VLH}-Cλ (or scFv_{VHL/VLH}-Cκ) of a different specificity and the joining (J) chain precursor expression plasmid to generate bispecific IgMs. This design took advantage of antibody inter-chain disulfide bond formation to generate uniform species of a bispecific antibody without sacrificing half of the antigen binding unit. There are eight possible combinations for each bispecific IgM. Various levels of pentamer expression were assessed. There was variability in the yield of the pentamer expression. The level of expression of the bispecific pentameric IgM appears to be influenced by the direction of VH→VL (or vice versa) and/or which scFv associated with the constant regions of the heavy (Cμ) or light chains (Cλ/κ).

In certain embodiments, binding of a first scFv of a bispecific IgM induces conformational changes within HIV envelope that opens up previously cryptic epitopes that are recognized by the second scFv. This can result in a tremendous synergy created by one pentameric IgM that has bispecific epitope specificity.

Also disclosed here are recombinant IgM compositions that target more than one epitope on the HIV envelope. Two different bispecific IgMs were successfully built and expressed. One example was the bispecific IgM—PGT121-VRC01-IgM. This composition incorporated PGT121 (targeting the V3 loop-associated glycans) and VRC01 (targeting the CD4 binding site on gp120) and the resulting bispecific IgM was termed PGT121-VRC01-IgM. Another bispecific IgM also built from PGT121 but partnered with PGDM1400 (targeting the V2 loop on gp120), giving rise to the bispecific IgM termed PGT121-PGDM1400-IgM. This recombinant composition has the specificity of a broadly neutralizing human IgG1 monoclonal antibody (bnmAb), PGT121, which recognizes a complex, carbohydrate dependent epitope related to the V3 loop of HIV gp120. The second specificity was derived from the human lgG1 bnmAb PGGM1400 that targets the V2 loop on HIV gp120. The resulting bispecific IgM was cloned, expressed, and purified. Its binding specificity was verified by competition assays. It depicts a monomer unit, five of which have been assembled to generate the pentameric lgM together with the J chain. The monomer was generated by grafting a single chain Fv representing the variable heavy (VH) and the variable light (VL) genes of the broadly neutralizing human IgG1 mAb PGT121. The VH and the VL fragments are separated by a linker. The binding partner on the light chain was generated by grafting the ScFv of VRC01, another broadly neutralizing anti-HIV mAb that recognizes a different target (the CD4 binding site). In this example, the order of VL and VH with regards to the constant portion of the λ chain (Cλ) has been reversed in the ScFv compared to that of PGT121. This order yielded optimal expression of the recombinant bispecific lgM in this particular example. The binding specificities of bispecific IgMs were verified through competitional ELISA against monospecific IgG1s.

### EXAMPLES

The following Examples are set forth to aid in the understanding of the embodiments of the invention, and are not intended and should not be construed to limit in any way the embodiments set forth in the claims which follow thereafter.

### Example 1—Generation of Class-Switched IgM Monoclonal Antibodies

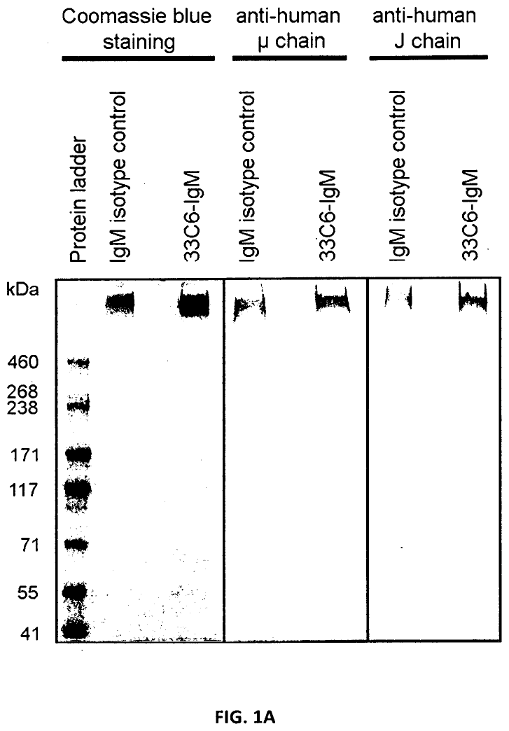

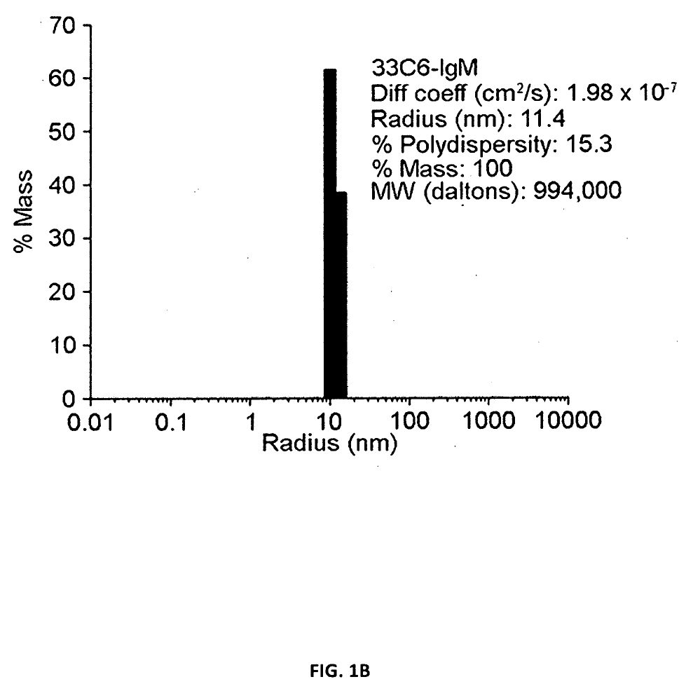

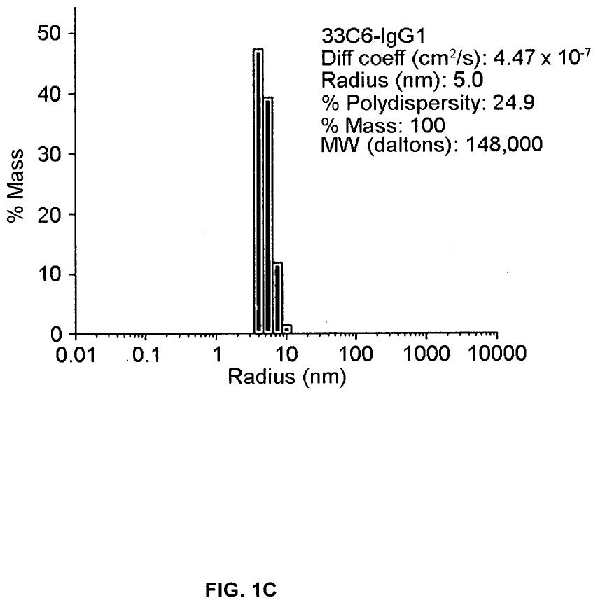

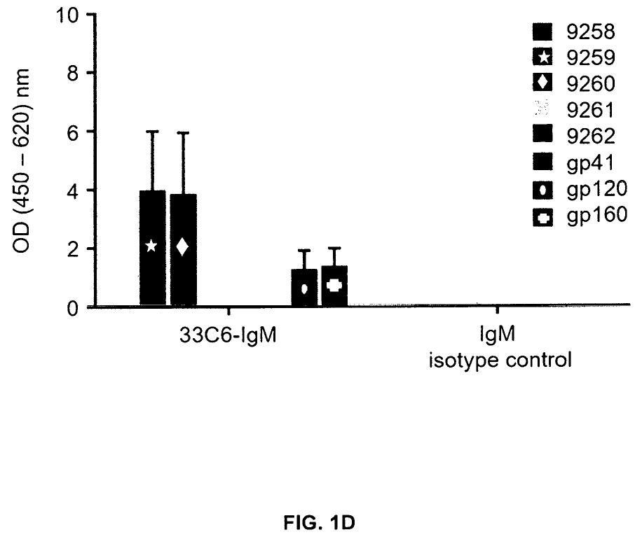

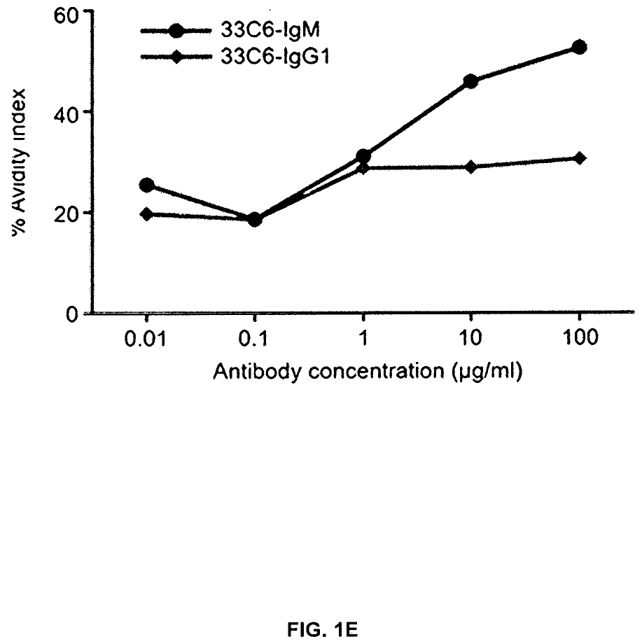

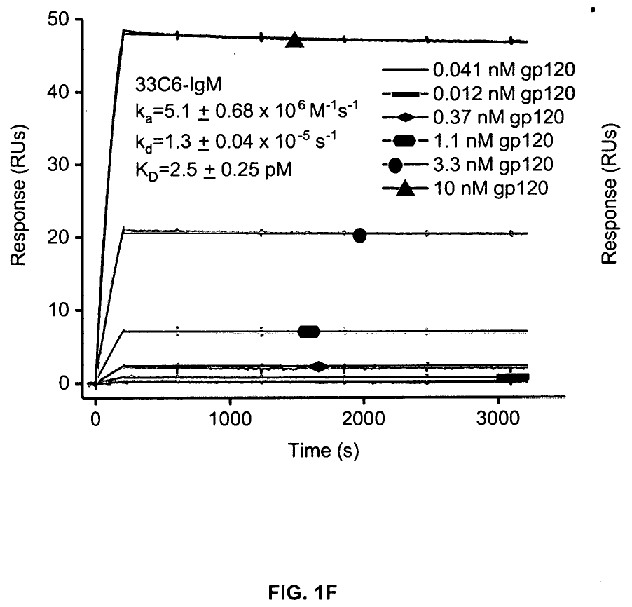

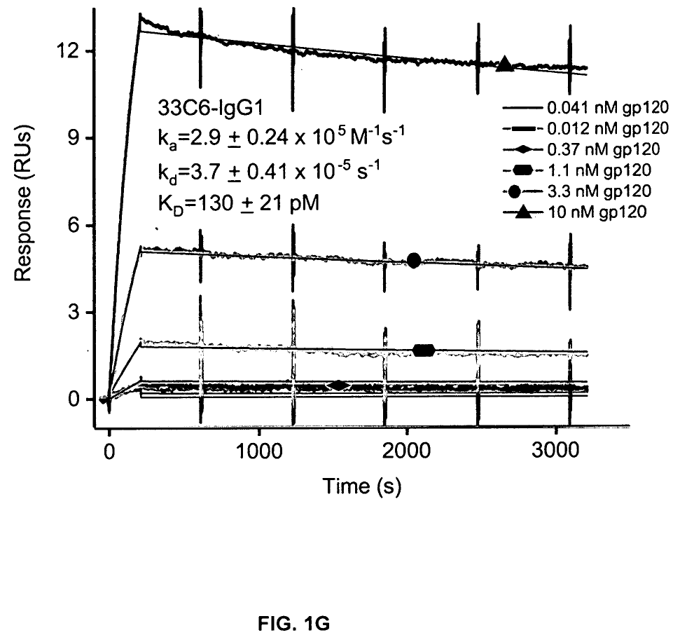

IgM antibodies directed to an epitope of an envelope protein of the HIV virus were generated. In an embodiment, the monoclonal IgG antibodies were MAb 33C6-IgG1 antibodies. These antibodies were directed to portions of the V3 loop of consensus HIV clade C gp120. To class-switch these antibodies to IgM, the heavy and light variable gene fragments were cloned in-frame with the human μ and λ chain constant regions, respectively. The 33C6-IgM antibodies were expressed by co-transfecting the resultant vector constructs with the human J chain precursor expression plasmid into Expi293 cells. The 33C6-IgM antibodies were purified from filtered culture supernatant with thiophilic resin affinity binding and cation exchange chromatography. The presence of polymeric 33C6-IgM was confirmed with denaturing, non-reducing sodium dodecyl sulfate-polyacrylamide gel electrophoresis (SDS-PAGE) and western blot analysis (FIG. 1A) and by DLS (FIGS. 1B-1C). FIG. 1A is a photographic image of a SDS-PAGE and western blot analysis for 33C6-IgM and 33C6-IgG1. FIG. 1B and FIG. 1C are graphical representations of the results from a dynamic light scattering assay to determine particle size of 33C6-IgM and 33C6-IgG1. Data are representative of 4 independent experiments.

The particle sizes of the purified 33C6-IgM and 33C6-IgG1 were measured by Dynamic Light Scattering as described in the methods here. The experiment was repeated 4 times on each sample, and representative data are shown. In the experiment shown, 100% of the mass of the IgM antibody was found in a particle with a diffusion coefficient of 1.98×10⁻⁷ cm²/s, a radius of 11.4 nm, 15.3% polydispersity, and a mass of 994,000 Da. The average mass from the 4 independent measurements was calculated as 994,000±23,000 Da. This is consistent with the MW expected for the pentameric particle, since the masses of 1 J chain, 10 heavy chains, and 10 light chains as calculated from the amino acid sequence is 874,707 Da, which is expected to be further increased by glycosylation. In the experiment shown, 100% of the mass of the IgG1 antibody was found in a particle with a diffusion coefficient of 4.47×10⁻⁷ cm²/s, a radius of 5.0 nm, 24.9% polydispersity, and a mass of 148,000 Da. The average mass from the 4 independent measurements was calculated as 148,000±3,600 Da. This is consistent with the MW expected for the monomeric particle, as the masses of 2 heavy chains and 2 light chains is 145,291 Da, which is expected to be further increased by glycosylation.

The 33C6-IgM recognized and bound to the conserved V3 loop crown of HIV Env as expected based upon the known epitope specificity of 33C6-IgG1 (FIG. 1D). FIG. 1D is a graphical representation of the 33C6-IgM binding to consensus HIV clade C peptides representing V3 and to Env proteins. 33C6-IgM bound with greater avidity to SHIV-1157ip gp120 than 33C6-IgG1 (FIG. 1E), confirming the known superior avidity of IgM. FIG. 1E is a graphical representation of the binding avidity of 33C6-IgM and 33C6-IgG1. Data are representative of two independent experiments. FIG. 1F is a graphical representation of the binding affinities of captured 33C6-IgM for solution phase SHIV-1157ip gp120, with representative concentration series of SPR sensorgrams ranging from 41 picomolar (pM) to 10 nanomolar (nM) of gp120. Global fits to a 1:1 binding model are overlaid in black. Average association rate constant (kₐ), dissociation rate constant (k_{d}), and equilibrium dissociation binding constant (K_{D}) with standard errors from 3 replicates are shown in the insert. FIG. 1G is a graphical representation of the binding affinities of captured 33C6-IgG1 for solution phase SHIV-1157ip gp120, with representative concentration series of SPR sensorgrams ranging from 41 picomolar (pM) to 10 nanomolar (nM) of gp120. Global fits to a 1:1 binding model are overlaid in black. Average kₐ, k_{d}, and K_{D} with standard errors from 3 replicates are shown in the insert. SPR analysis revealed that 33C6-IgM bound to SHIV-1157ip gp120 with a K_{D} of 2.5 pM (FIG. 1F). In contrast, 33C6-IgG1 had a K_{D} of 130 pM (FIG. 1G), indicating that the IgM class bound significantly tighter than the IgG1 isotype of mAb 33C6. The on-rate for 33C6-IgM was 18-fold faster than that of the IgG1 version, and the IgM off-rate was 2.8 times slower than that of 33C6-IgG1. Together, these parameters account for the much tighter binding of the IgM isoform to soluble gp120 of the challenge virus.

### Example 2—33C6-IgM Neutralized and Captured SHIV In Vitro

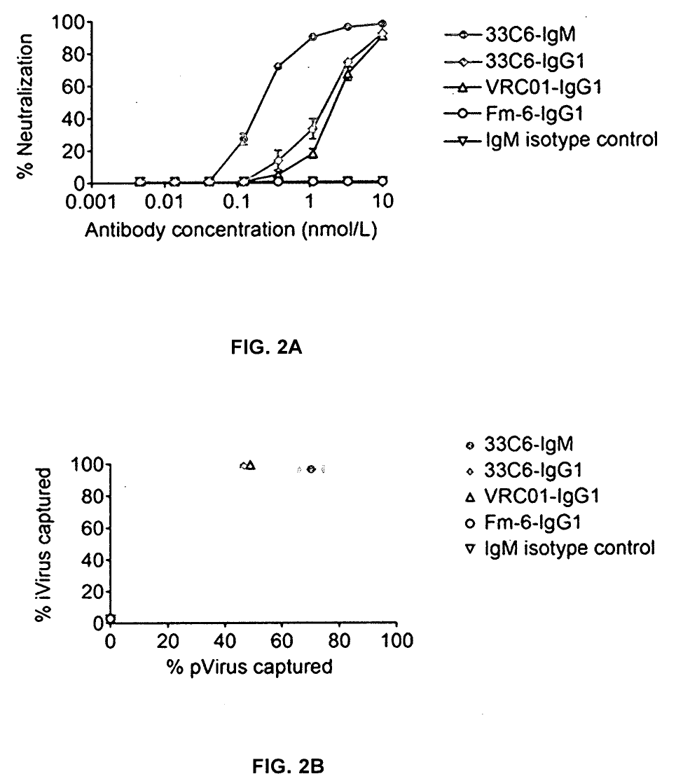

To assess the potential of 33C6-IgM to protect RMs against mucosal SHIV challenge—the neutralization of SHIV-1157ipEL-p, the intended challenge virus strain, by 33C6-IgM and the IgG1 isotype was tested by TZM-bl assay. This R5 clade C tier 1 SHIV strain had been used to demonstrate complete cross-clade protection of RMs by the human mAb HGN194. The latter has a similar epitope specificity as the 33C6 mAbs. The 33C6-IgM antibodies neutralized SHIV-1157ipEL-p 10× better compared to 33C6-IgG1 antibodies (FIG. 2A). FIG. 2A is the graphical representation of the neutralization of the challenge virus, SHIV-1157ipEL-p, by 33C6-IgM and 33C6-IgG1. Of note, the pentameric IgM contains 5× more antigen binding sites compared to IgG1 at the same molar concentration, which might explain the greater neutralization potency of 33C6-IgM.

Next, virion capture assays with the two 33C6 mAbs were performed. The virion capture correlates with protection against mucosal SHIV-1157ipEL-p challenge. The IgM form depleted physical virus particles by 75% and infectious virions by 96% (FIG. 2B). As compared to 33C6-IgG1, the IgM form depleted more physical particles. Importantly, both isoforms removed almost all infectious virions. FIG. 2B is the graphical representation of the capture of physical virus (pVirus) and infectious virus (iVirus) particles by 33C6-IgM and 33C6.-IgG1. Data are representative of two independent experiments. Error bars, mean±SEM. VRC01-IgG1 was used as positive control, while Fm-6-IgG1 and IgM isotype control were used as negative controls. Because a different secondary anti-IgM capture antibody was required for 33C6-IgM to capture cell-free virus using Protein G micro-beads, virion capture by 33C6-IgM could not be compared directly to that of 33C6-IgG1.

### Example 3—33C6-IgM Protected RMs Against High-Dose Mucosal SHIV Challenge

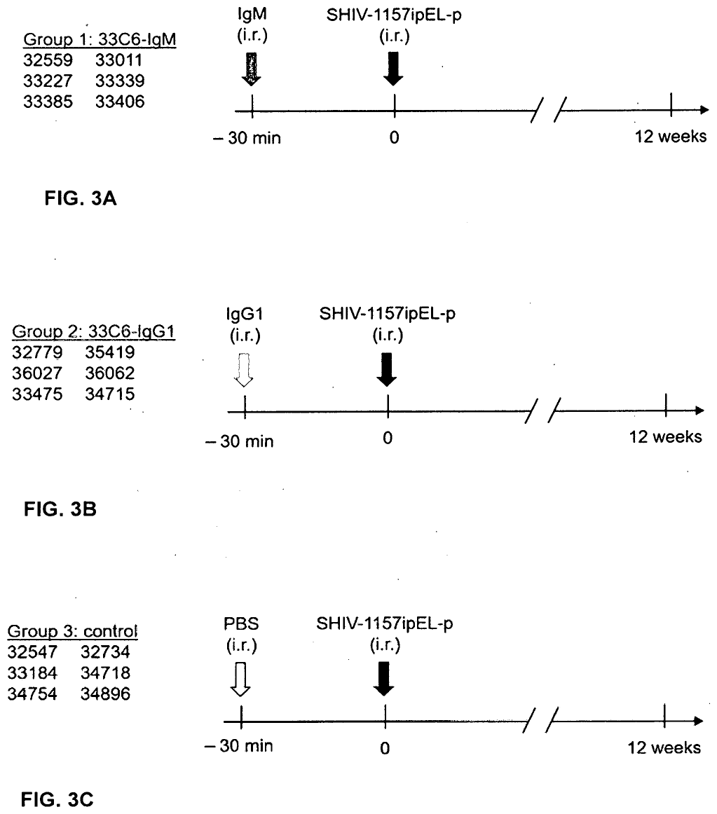

The experimental design of infection of the Rhesus macaques is shown in FIGS. 3A-3C. All primate studies were conducted in strict accordance with the recommendations in the Guide for the Care and Use of Laboratory Animals of the U.S.A.

The animals were randomized into three groups (n=6 per group). SHIV-1157ipEL-p challenge with 31.5 50% animal infectious doses (31.5 AID₅₀) was given to all three groups. FIG. 3A shows the timeline of the study, where Group 1 received 33C6-IgM intrarectally (i.r.) (Red arrow). FIG. 3B shows the timeline of the study, where Group 2 was given 33C6-IgG1 i.r. (blue arrow). FIG. 3C shows the timeline of the study, where Group 3 received only phosphate-buffered saline (PBS) i.r. (empty arrow). Groups 1 and 2 were given i.r. mAbs at a total dose of 1.25 mg in 2.1 ml of PBS. Group 1 RMs received 33C6-IgM (1.26 nmol); Group 2 received 33C6-IgG1 (8.45 nmol). Group 3 (controls) received 2.1 ml of PBS i.r. only. 33C6-IgM, 33C6-IgG1 or PBS was given 30 min before the virus challenge. In each of FIGS. 3A-3C, the black arrow indicates the time when a high-dose i.r. Thirty minutes after delivery of passive immunization, all RMs were challenged intra-rectally with 31.5 50% animal infectious doses (AID₅₀) of SHIV-1157ipEL-p, and plasma viral RNA (vRNA) levels were monitored for 12 weeks. Plasma samples for mAb detection and viral load determination were obtained on the day of SHIV challenge and prospectively thereafter. Plasma viral RNA levels were measured as described. Statistical analyses were performed using GraphPad Prism version 5 for Windows (GraphPad Software Inc.). The time-to-peak viremia was analyzed by Kaplan-Meier analysis using the log-rank test with Holm-Sidak adjusted two-sided p-values.

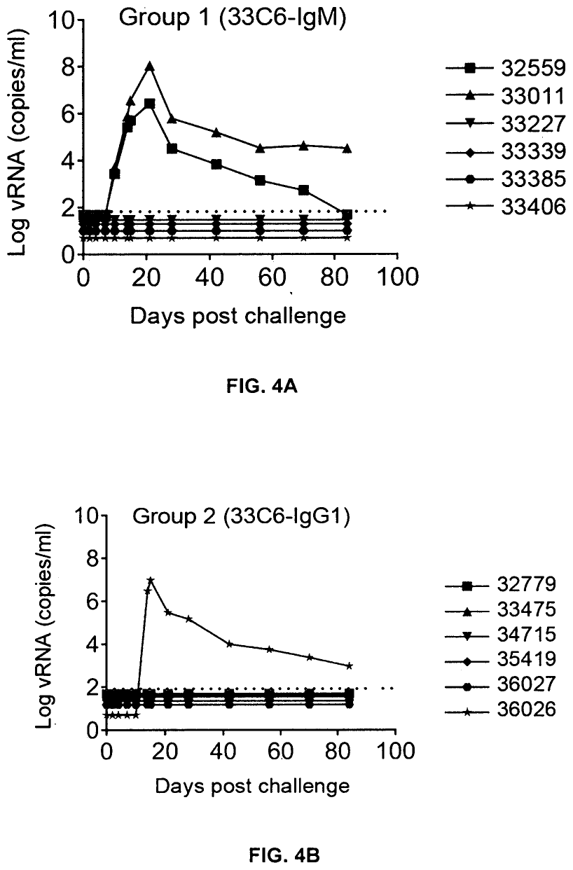

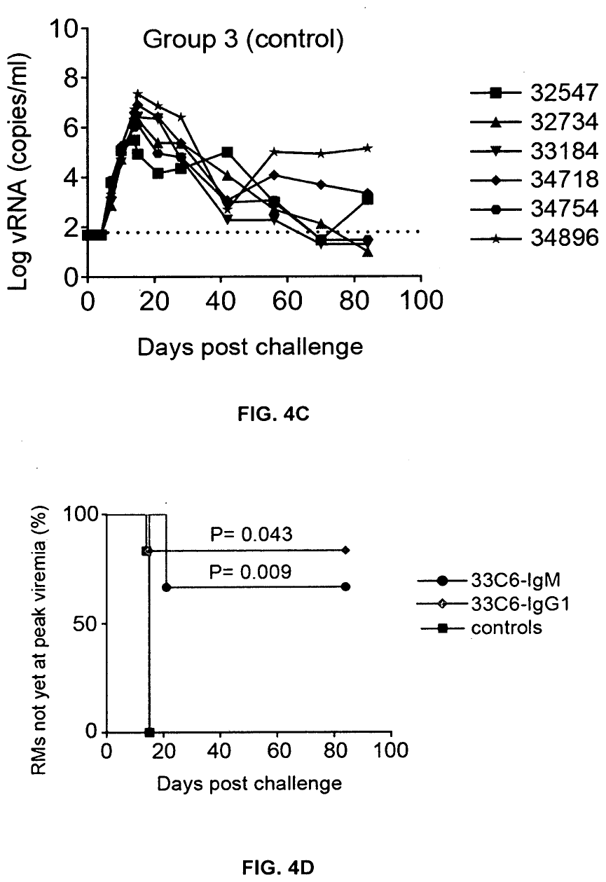

FIGS. 4A-4D are graphical representations of the plasma viral loads in macaques challenged with SHIV-1157ipEL-p. Plasma viral RNA is expressed as a log scale of the viral RNA copies per milliliters (log vRNA copies/ml). FIG. 4A shows the plasma viral loads in macaques in Group 1, who received 33C6-IgM. FIG. 4B shows the plasma viral loads in macaques in Group 2, who received 33C6-IgG1. Four out of six (67%) RMs in Group 1 (33C6-IgM) (FIG. 4A), and five out of six (83%) RMs in Group 2 (33C6-IgG1) (FIG. 4B) remained aviremic. FIG. 4C shows the plasma viral loads in macaques in Group 3 (controls). In contrast, all control RMs became systemically infected by week 2 with a median peak viremia of 10⁶ vRNA copies/ml (FIG. 4C); the three RMs with breakthrough infection in Groups 1 and 2 had peak vRNA levels >10⁶ copies/ml. In each of FIGS. 4A-4C, the black dotted line indicates the lower limit of viral RNA detection (50 copies/ml).

FIG. 4D is a graphical representation of the Kaplan-Meier analysis of time until peak viremia for all three Groups. Log-rank test was used to determine significance. The time to peak viremia was analyzed by Kaplan-Meier analysis using the log-rank test with Holm-Sidak adjusted two-sided p-values (FIG. 4D). Passively immunized RMs in Groups 1 and 2 had delayed peak viremia compared to Group 3 controls (Group 1, p=0.009; and Group 2, p=0.043). Of note, the 1.25 mg mAb dose used in this study contains 5-fold fewer IgM than IgG1 molecules. Thus, at equimolar concentrations, IgM might be better than IgG1. Taken together, data demonstrate that IgM is as effective as IgG1 in protecting against mucosal SHIV acquisition.

### Example 4—33C6-IgM Treatment Accelerated the Induction of Anti-SHIV Neutralizing Antibodies

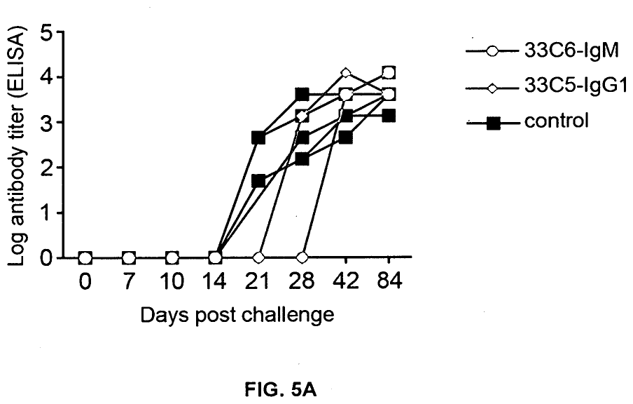

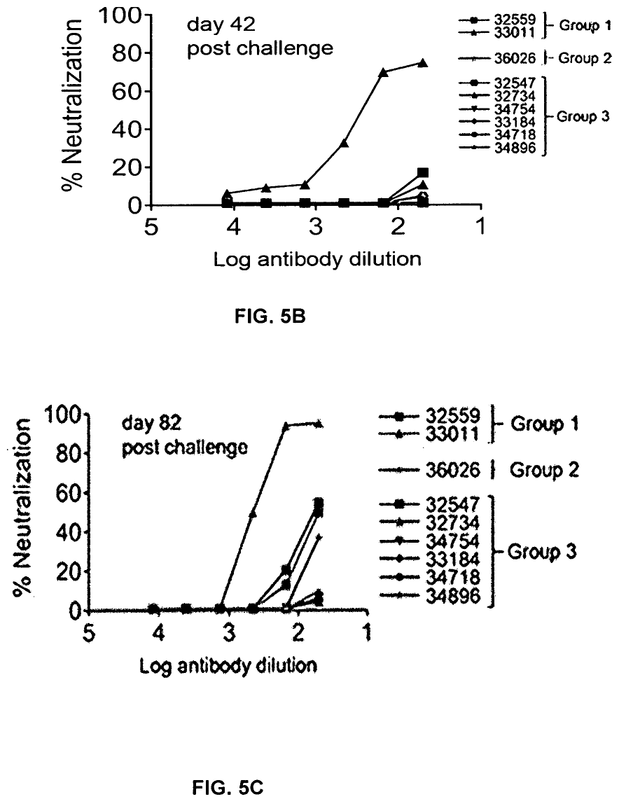

The development of SHIV-specific antibodies in RMs with breakthrough infection were examined. All infected RMs seroconverted (FIG. 5A); in passively immunized RMs of Groups 1 and 2 that became infected, anti-Env antibodies became detectable with delays. The plasma samples from all RMs with systemic SHIV infection were evaluated for neutralizing antibodies against the SHIV-1157ipEL-p challenge virus (FIGS. 5B and C). Among day 42 plasma samples, neutralizing antibodies were only seen in one RM, animal 33011 treated with 33C6-IgM (FIG. 5B). By day 84, plasma from both of the infected, IgM-treated RMs neutralized the challenge SHIV by ≥50% (FIG. 5C). In contrast, neutralizing antibodies were present in only one out of six controls, indicating a trend for earlier development of autologous neutralizing antibodies in the IgM-treated RMs with breakthrough infection (p=0.1). Mucosal administered IgM protects against high-dose mucosal SHIV challenge and may accelerate the development of autologous neutralizing antibodies in case virus acquisition is not prevented.

### Example 5—Expanding the Effector Function of IgM

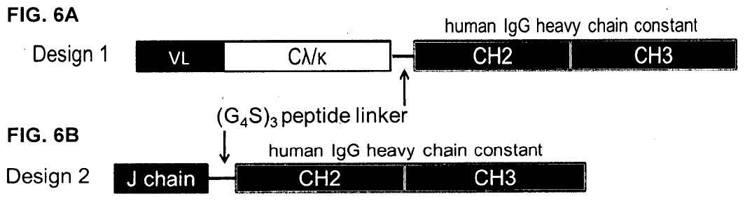

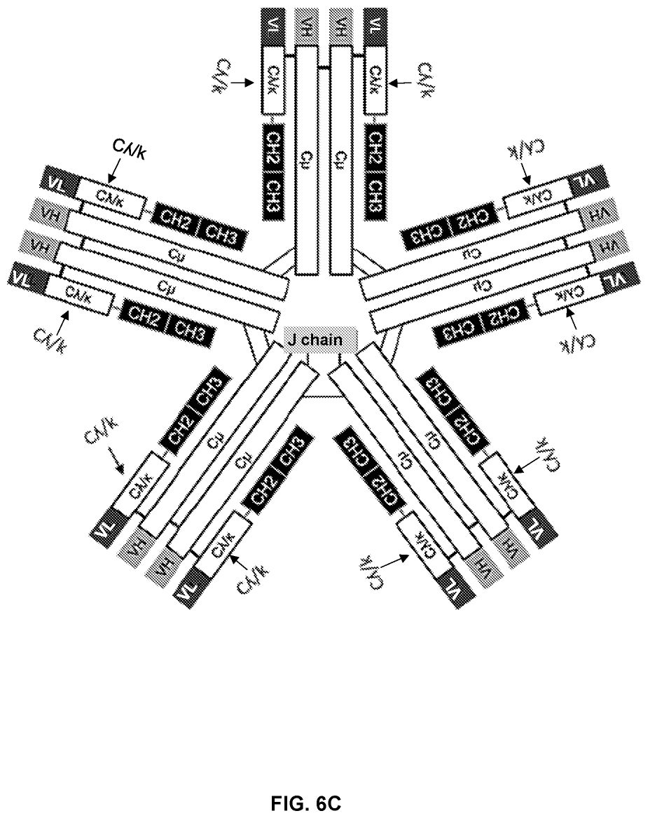

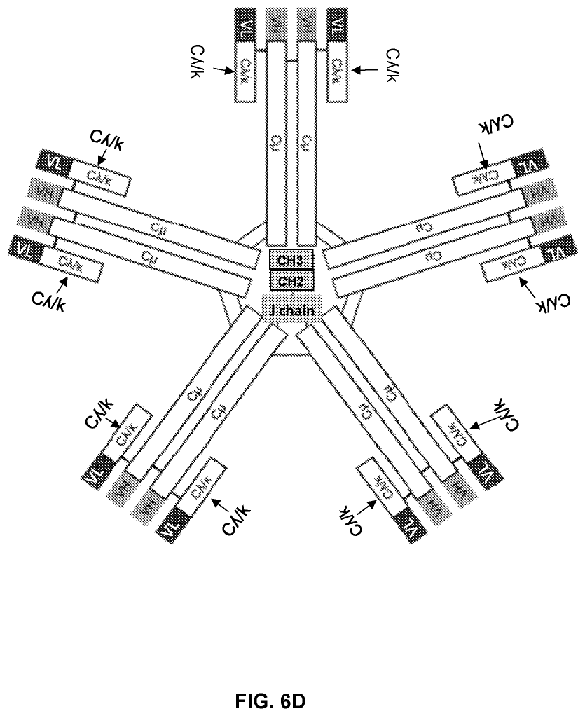

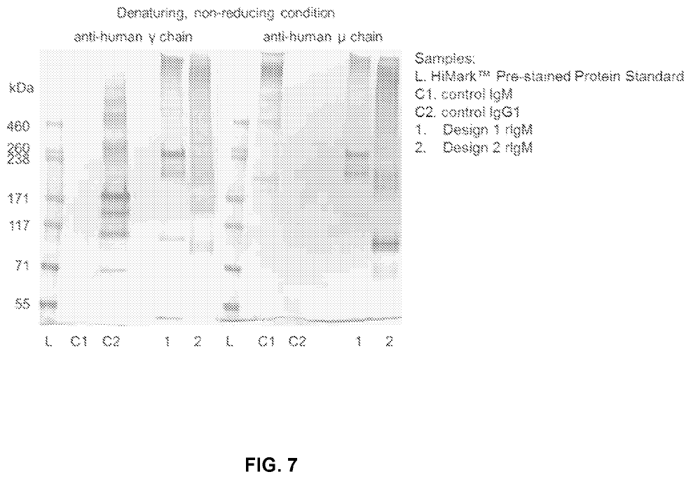

As shown in FIG. 6A, the human IgG constant heavy chain CH2-CH3 regions were fused via three repeats of the tetra-glycine-serine peptide linker (G₄S)₃ to the C-terminus of the light chain constant region. The first 112 amino acids of the human IgG1 heavy chain constant were excluded to remove all of the cysteines associated with inter-chain disulfide bond formation in order to prevent Fcγ dimerization with other chains. This embodiment allows ten copies of Fcγ to be incorporated into every pentameric rIgM. In FIG. 6A, the human IgG constant heavy chain CH2-CH3 regions were fused via three repeats of the tetra-glycine-serine peptide linker (G₄S) to the C-terminus of the light chain constant region. This strategy allows ten copies of Fcγ to be incorporated into every pentameric rIgM, as shown in FIG. 6C. To express this IgM-Fcγ composition, Expi293 cells were co-transfected with three different plasmids encoding (i) the IgM heavy chain, (ii) the J chain, and (iii) a recombinant altered light chain containing the CH2-CH3 region of the human IgG heavy chain constant region fused to the C-terminus of the light chain constant region via the (G₄S)₃ linker. Culture supernatants were harvested and tested for the presence of Fcγ in pentameric IgM, as shown in FIG. 7.

As shown in FIG. 6B, the CH2-CH3 region of the human IgG heavy chain constant region was cloned to the C-terminus of the joining (J) chain through the (G₄S)₃ linker described above. This recombinant composition contains only one Fcγ per pentameric IgM molecule given that pentameric IgM contains just one J chain. In FIG. 6B, the CH2-CH3 region of the human IgG heavy chain constant region was connected to the C-terminus of the Joining (J) chain through the same (G₄S)₃ linker described in FIG. 6A. This design is expected to yield only one Fcγ per pentameric IgM molecule given that pentameric IgM contains just one J chain, as shown in FIG. 6D. To express this IgM-Fcγ composition, Expi293 cells were co-transfected with three different plasmids encoding (i) an IgM heavy chain expression plasmid, (ii) the corresponding light chain expression plasmid, and (iii) the expression plasmid with the altered J chain—the CH2-CH3 region of the human IgG heavy chain constant region cloned to the C-terminus of the J chain through the (G₄S)₃ linker. Culture supernatants were harvested and tested for the presence of Fcγ in pentameric IgM, as shown in FIG. 7.

FIG. 7 is a photographic image of a SDS-PAGE and western blot analysis for the presence of Fcγ in pentameric IgM compositions corresponding to FIGS. 6C and 6D. By transient transfection assay followed by transient expression, IgM containing sequences from Fcγ were expressed at lower levels compared to the parental IgM. Experimental conditions and constructs can be optimized for the desired expression level of the Fcγ containing IgM.

### Example 6—Bispecific IgM Design and Expression

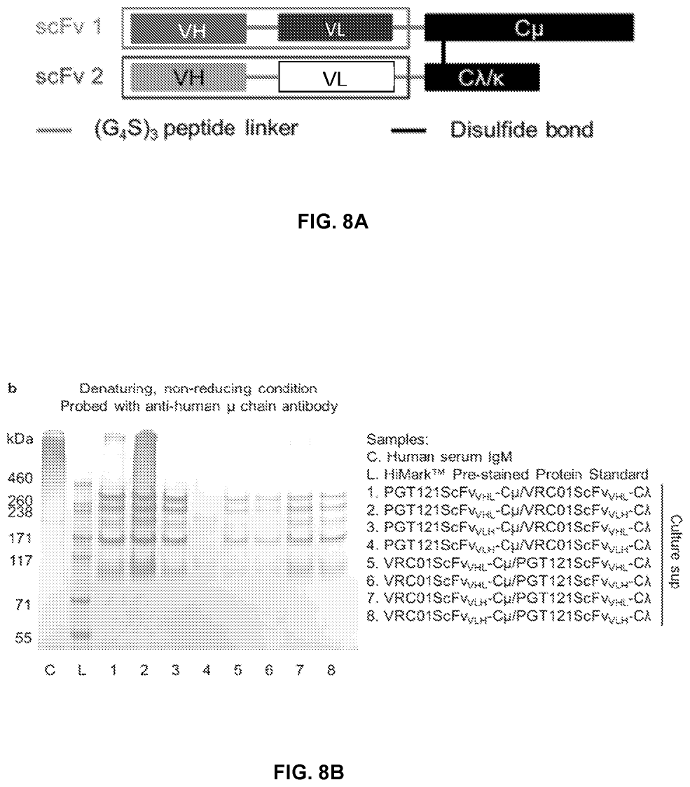

The heavy (VH) and light (VL) chain variable genes of anti-HIV envelope (Env) monoclonal antibodies (mAbs) were cloned. Single-chain variable fragment (scFv) molecules were generated, which utilized 3 repeats of the tetra-glycine-serine peptide linker (G₄S)₃ to connect the VH and VL segments. The scFvs of mAbs in either the VH→VL (scFvVHL) or VL→VH (scFvVLH) direction were linked to the heavy chain constant region (Cμ) or light chain constant region (Cλ or Cκ) through (G₄S)₃ to generate scFvVHL/VLH-Cμs and scFvVHL/VLH-Cλs (or scFvVHL/VLH-Cκs). The plasmid expressing scFvVH/VLH-Cμ of a given specificity was co-transfected with a plasmid expressing scFvVHL/VLH-Cλ (or scFvVHL/VLH-Cκ) of a different specificity and the joining (J) chain precursor expression plasmid to generate bispecific IgMs (FIG. 8A). FIG. 8A is a schematic presentation of bispecific IgM heavy and light chain. The direction of VH and VL can be switched within each scFv. The position of scFv 1 and scFv 2 are interchangeable as well. This design took advantage of antibody inter-chain disulfide bond formation to generate uniform species of a bispecific antibody without sacrificing half of the antigen binding unit. There are eight possible combinations for each bispecific IgM. Various levels of pentamer expression were assessed, as shown in FIG. 8B. FIG. 8B is a photographic image of a SDS-PAGE gel with the eight combinations and controls, probed with anti-human μ chain antibody for the presence of a total of eight combinations of heavy and light chain plasmids for a bispecific IgM, PGT121-VRC01-IgM.

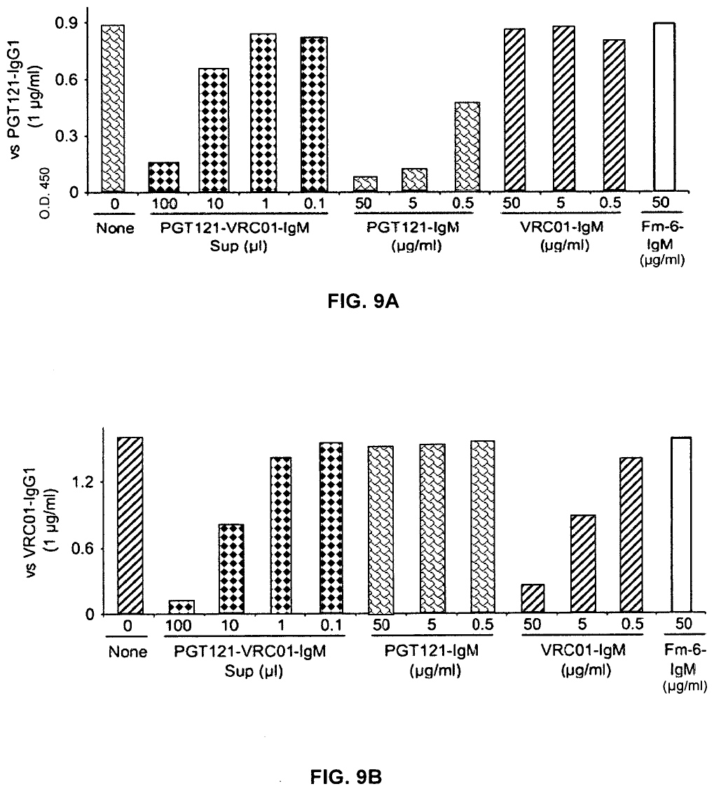

An ELISA plate was coated with HIVBaL gp120 (1 μgimp. After blocking with 1% bovine serum albumin (BSA), the mAbs were mixed with (a) PGT121-IgG1 or (b) VRC01-IgG1 The mixtures were added to the plate and incubated at 37° C. for 2 h. After washing away unbound Abs, a horseradish peroxidase-conjugated goat anti-human Fcγ Ab was added to label gp120-bound IgG1 mAbs. Finally, 3,3′,5,5′-Tetramethyl-benzidine (TMB) substrate and stop solutions were added and absorbance was read at 450 nm. Fm-6-IgM, negative isotype control. The secondary Ab did not react with any IgM mAbs (data not shown). FIGS. 9A and 9B are graphical representations of the competitive binding assays to verify epitope specificity of the bispecific IgM, PGT121-VRC01-IgM using two mAbs: PGT121-IgG1 and VRC01-IgG1, respectively. The data shows the expected specificities of the bispecific IgM.

### Methods

### Cell Lines, Reagents and Virus

TZM-bl cells were obtained through the NIH AIDS Reagent Program, Division of AIDS, NIAID, from J. C. Kappes, X. Wu and Tranzyme Inc. SHIV-1157ip gp120 was prepared as previously described. SHIV-1157ipEL-p stock (grown in RM peripheral blood mononuclear cells (PBMC)) had a p27 concentration of 792 ng/ml and 7.8×105 50% tissue culture infectious doses (TCID50)/ml (measured in TZM-bl cells).

### Preparation of 33C6 mAbs

We previously described the production of 33C6-IgG1 mAb; 33C6-IgM mAb was prepared as follows. Human μ chain constant region and human λ chain constant region were PCR amplified from pFUSEss-CHIg-hM and pFUSE2-CLIg-h12 (InvivoGen) using Q5 High-Fidelity 2× Master Mix (NEB) following the manufacturer's recommendation. Following gel purification, PCR products were assembled with a Kozak-murine Ig leader sequence and cloned downstream of a CMV promoter in a pcDNA3.4 plasmid (ThermoFisher Scientific) using NEBuilder HiFi DNA Assembly Master Mix (NEB). The resulting plasmids were designated pTBRI-hM and pTBRI-hl, respectively. 33C6 heavy chain and light chain variable gene fragments were PCR amplified from the 33C6-IgG1 expression plasmids and cloned into pTBRI-hM and pTBRI-hl to yield plasmids pTBRI-33C6-hM and pTBRI-33C6-hl, respectively. Full-length 33C6-IgM mAb was expressed in Expi293F cells (ThermoFisher Scientific) transiently co-transfected with pTBRI-33C6-hM, pTBRI-33C6-hl and human J chain precursor expression plasmids using ExpiFectamine 293 Transfection Kit (ThermoFisher Scientific). Cells were maintained in Expi293 expression medium (ThermoFisher Scientific) for 4 days at 37° C., 8% CO₂ with continuous shaking at 135 rpm. Antibody was purified from filtered supernatants using Thiophilic Resin (G-Biosciences) followed by HiTrap SP HP column separation (GE Healthcare Life Sciences). Purity and polymeric state of 33C6-IgM was verified under denaturing and non-reducing polyacrylamide gel electrophoresis (NuPAGE™ 3-8% Tris-Acetate Protein Gel, ThermoFisher Scientific). The presence of human μ and J chains was verified by western blot with horse-radish peroxidase (HRP)-conjugated goat anti-human IgM, Fc5μ fragment-specific antibody (Jackson ImmunoResearch) and J-chain antiserum (InvivoGen) in conjunction with HRP-conjugated donkey anti-rabbit IgG(H+L) (Jackson ImmunoResearch), respectively.

### Dynamic Light Scattering Studies

The particle sizes of the purified 33C6-IgM and 33C6-IgG1 were measured by Dynamic Light Scattering in PBS at 24° C. using a Protein Solutions DynaPro (Wyatt Technology) and analyzed with Dynamics software (Wyatt Technology). The predicted molecular weight of 33C6-IgM and 33C6-IgG1 was calculated based on the amino acid sequences using compute pI/Mw tool at ExPASy Bioinformatics Resource Portal (SIB Swiss Institute of Bioinformatics; https://web.expasy.org/compute_pi/).

### Surface Plasmon Resonance (SPR) Studies

SPR studies were carried out utilizing a Biacore T200 instrument on a CM5 chip in HBS-EP+ running buffer (10 mM Hepes, 150 mM NaCl, 3 mM EDTA, 0.05% surfactant P20) at 25° C. An anti-human IgM Fc5μ fragment-specific capture antibody (Jackson ImmunoResearch) was immobilized on FC1 and FC2 utilizing an amine coupling kit (GE Healthcare). An anti-human IgG Fc capture antibody from a human IgG capture kit (GE Healthcare) was immobilized on FC3 and FC4 utilizing an amine coupling kit (GE Healthcare). 33C6-IgM was captured on FC2, and 33C6-IgG1 was captured on FC4. A concentration series of SHIV-1157ip gp120 ranging from 4.6 pM to 10 nM was flowed over all 4 surfaces at 100 μl/min, interspersed with buffer blanks for double referencing. During each cycle, gp120 was injected for 220 s, followed by a change to running buffer for 3000 s. FC1 and FC2 were regenerated by an injection of 10 mM glycine pH 1.7 for 180 s at 20 μl/min, while FC3 and FC4 were regenerated by an injection of 3 M MgCl₂ for 30 s at 20 μl/min. Data were double referenced, first by subtracting the reference cell data from the experimental cell data (i.e., FC2-FC1) and (FC4-FC3), and then by subtracting the buffer blanks. Overlay plots were made for all curves from 41 pM-10 nM, and data were globally fit to a 1:1 binding model utilizing Biacore T200 evaluation software v. 2.0 (GE Healthcare), from which the association rate constant kₐ, the dissociation rate constant k_{d}, and the equilibrium dissociation constant K_{D} were derived. Average values for each quantity were determined from 3 replicates, and average values and standard error were calculated utilizing OriginPro 2017 Software (OriginLab Corp.), and are indicated directly on FIG. 1C and FIG. 1D. The fit parameters for the IgM data set shown in FIG. 1C are kₐ=4.0×10⁶ M⁻¹s⁻¹, k_{d}=1.2×10⁻⁵ s⁻¹, K_{D}=2.9 pM, chi²=0.030 RU². The fit parameters for the IgG1 data set shown in FIG. ID are kₐ=3.0×10⁵ M⁻¹s⁻¹, k_{d}=4.3×10⁻⁵ s⁻¹, K_{D}=146 pM, chi²=0.042 RU².

### Animals

Eighteen adult, outbred, naïve, male, Indian-origin RMs (Macaca mulatta) were housed at the SNPRC, San Antonio, Tex. SNPRC, a facility fully accredited by the Association for Assessment and Accreditation of Laboratory Animal Care International, adheres to the Guide for the Care and Use of Laboratory Animals. All procedures were approved by the Animal Care and Use Committee of Texas Biomedical Research Institute, SNPRC's parent institution. All RMs were negative for Mamu-B*17 allele associated with spontaneous virologic control; one RM 32547 (randomized to the control group) was positive for the Mamu-B*08 allele also associated with spontaneous virologic control. Similarly, RMs with the Mamu-A*01 allele that contributes to spontaneous virologic control were evenly distributed in each group. RMs were randomized into groups (n=6 per group).

### Plasma vRNA Loads

RNA was isolated by QIAamp Viral RNA Mini-Kits (Qiagen), and vRNA levels were measured by quantitative reverse-transcriptase polymerase chain reaction (qRT-PCR) for SIV gag sequences. Assay sensitivity was 50 vRNA copies/ml. Time to first detection of viremia was analyzed by Kaplan-Meier analysis.

### ELISAs

Plasma mAb binding to SHIV-1157ip gp120 was evaluated by ELISA as described [1]. Briefly, plates were coated with monomeric SHIV-1157ip gp120 (1 μg/ml) in 100 μl carbonate buffer (0.05 M carbonate-bicarbonate buffer, pH 9.6, Sigma) overnight at 4° C., washed 3× with 0.05% Tween 20 in PBS (0.05% PBS/T), and blocked with 4% non-fat milk in PBS for 1 h at 37° C. One hundred μl of heat-inactivated plasma diluted serially in dilution buffer (1% non-fat milk in PBS) were added to duplicate wells and incubated for 2 h at 37° C. Plates were washed 3× in 0.05% PBS/T, and binding was detected with HRP-conjugated rabbit anti-monkey IgG (whole molecule) (Sigma) antibody. After 1 h of incubation at 37° C., 3,3′,5,5′-Tetramethylbenzidine (TMB) single solution (ThermoFisher Scientific) was added, and the reaction was terminated by the addition of 1 N H₂SO₄. Plates were read at 450 nm by a Mithras LB 940 Multimode Microplate Reader (Berthold Technologies). Antibody titers were calculated as the reciprocal sample dilution giving optical density (OD)>mean+5× standard deviation of background (pre-immune samples) at the same dilution.

Epitope binding specificity for 33C6-IgM mAbs was determined by ELISA with consensus clade C peptides (NIH AIDS Research and Reference Reagent Program) performed as described above. Briefly, plates were coated with corresponding peptides (5 μg/ml) in triplicates, blocked and probed with various concentration of 33C6-IgM or human serum IgM (Sigma). To detect binding, plates were probed with HRP-conjugated goat anti-human IgM antibody (Jackson ImmunoResearch). The sequences of the various peptides used were as follows: peptide 9258, VEIVCTRPNNNTRKS; peptide 9259, CTRPNNNTRKSIRIG; peptide 9260, NNNTRKSIRIGPGQT; peptide 9261, RKSIRIGPGQTFYAT and peptide 9262, RIGPGQTFYATGDII.

### Avidity Assay

Analysis of antibody avidity was performed as described [4]. Briefly, ELISA plates were coated with 50 μg/ml concanavalin A (Sigma) for 1 h at room temperature. After overnight incubation with 2 μg/ml monomeric SHIV-1157ip gp120 at 4° C., plates were blocked with 3% N-Z-Case plus (Sigma) 0.5% PBS/T for 1 h at 37° C., then incubated with 50 μl of serial dilutions of mAbs in two sets of triplicate wells for 1 h at 37° C. One hundred μl of 8 M urea solution (Sigma) was added to one set of the triplicate wells, and 100 μl of PBS was added to the other set of triplicate wells and incubated 3× (5 min each with wash steps in between) at 37° C. Then wells were washed thoroughly with 0.05% PBS/T and incubated with HRP-conjugated goat anti-human IgG1 or HRP-conjugated goat anti-human IgM (Jackson ImmunoResearch). Finally, the colorimetric reaction was developed with TMB single solution (ThermoFisher Scientific) and terminated with 1 N H₂SO₄. Avidity index (AI) was calculated as follows:

Al(%)=(OD₄₅₀ nm of samples washed with 8 M urea/OD₄₅₀ nm of samples washed with PBS)×100.

### Neutralization Assay

Neutralization of SHIV-1157ipEL-p by 33C6 mAbs or antibodies in RM plasma was determined using the TZM-bl assay as described [5]. Briefly, virus was incubated with serially diluted mAbs or plasma for 1 h at 37° C. TZM-bl cells (5×10³/well) and DEAE-dextran (Sigma) were added to the virus without antibody (baseline) or with antibodies. After incubation for 48 h at 37° C., luciferase activity was quantified in a CentroPRO LB 962 Budget Microplate Luminometer (Berthold Technologies) upon addition of Bright-Glo luciferase assay substrate (Promega). VRC01-IgG1 was used as positive control; IgM isotype control (ThermoFisher Scientific) and Fm-6-IgG1 were used as negative controls. Percentage neutralization was calculated relative to baseline luciferase activity or luciferase activity level of pre-immune samples for 33C6 mAb or RM plasma sample neutralization, respectively. Neutralizing antibody titers were estimated as the reciprocal serum dilution giving 50% inhibition of virus replication.

### Virion Capture Assay (VCA)

We performed the VCA as follows. Briefly, 10 μg/ml of 33C6 mAbs were coincubated with 10⁸ vRNA copies/ml of SHIV-1157ipEL-P (in 200 μl reaction) for 1 h at 37° C. to form antibody-virion immune complexes (ICs). To enable capture of IgM-virus complexes by the Protein G micro beads (Miltenyi Biotec), the ICs were incubated for additional 1 h at 37° C. in the presence of 20 μg/ml goat anti-human IgM antibody (Jackson ImmunoResearch). After ICs were mixed with Protein G micro beads for 30 min at 37° C., the mixture was loaded to a μ Column (Miltenyi Biotec) under a magnetic field and washed. Unbound free virions were collected in the flow-through and measured by Gag p27 ELISA (Advanced BioScience Laboratories). The infectivity of the flow-through virus was evaluated by TZM-bl assay. A virus-only control (no antibody) was used to set maximum limit of infectivity; VRC01-IgG1 was used as positive control; IgM isotype control (ThermoFisher Scientific) and Fm-6-IgG1 were used as negative controls.

The percentage of total physical virus particles captured (pVirion) was calculated as:

pVirion=[1−(p27 concentration of virus with mAbs in flow-through)/(p27 concentration of virus in flow-through of virus-only control)]×100%.

The percentage of captured infectious virions (iVirion) was calculated as:

iVirion=[1−(flow-through infectivity of virus with mAbs)/(flow-through infectivity of virus-only control infectivity)]×100%.

Those skilled in the art will recognize, or be able to ascertain using no more than routine experimentation, many equivalents to the specific embodiments of the invention described herein. Such equivalents are intended to be encompassed by the following claims.

What is claimed is:

1. A method of inducing an immune response directed towards preventing or reducing the risk of a human immunodeficiency virus (HIV) infection in a mammalian subject, comprising
administering to the mammalian subject an effective amount of a composition containing immunoglobulin M (IgM) antibodies directed to an epitope of a human immunodeficiency virus envelope protein.

2. The method of claim 1, wherein the composition containing IgM antibodies is formulated for a mucosal administration.

3. The method of claim 2, wherein the mucosal layer is a rectal mucosal layer.

4. The method of claim 1, wherein the human immunodeficiency virus envelope protein is HIV-1 gp120.

5. A recombinant immunoglobulin M composition, comprising:
a Fcγ fragment of an immunoglobulin G connected to a carboxy terminus of a joining chain of an immunoglobulin M.

6. The recombinant immunoglobulin M composition of claim 5, wherein the Fcγ fragment of the immunoglobulin G is connected to the carboxy terminus of the joining chain of the immunoglobulin M via a linker.

7. The recombinant immunoglobulin M composition of claim 6, wherein the linker is a glycine- and serine-rich linker.

8. A recombinant immunoglobulin M composition, comprising:
a Fcγ fragment of an immunoglobulin G connected to a carboxy terminus of a constant region of a light chain of immunoglobulin M.

9. The recombinant immunoglobulin M composition of claim 8, wherein the Fcγ fragment of the immunoglobulin G is connected to the carboxy terminus of the constant region of the light chain of the immunoglobulin M via a linker.

10. The recombinant immunoglobulin M composition of claim 9, wherein the linker is a glycine- and serine-rich linker.

11. A recombinant immunoglobulin M composition, comprising:
a plurality of monomers of a bispecific antibody containing:
a first constant region linked to a first variable region, wherein the first variable region includes a variable region of a first heavy chain and a variable region of a first light chain capable of specifically binding to a first epitope of a pathogen; and
a second constant region linked to a second variable region, wherein the second variable region includes a variable region of a second heavy chain and a variable region of a second light chain capable of specifically binding to a second epitope of the pathogen.

12. The recombinant immunoglobulin M composition of claim 11, wherein the first epitope and the second epitopes are two different epitopes of a human immunodeficiency virus envelope protein.

13. The recombinant immunoglobulin M composition of claim 11, wherein the first epitope is from a first human immunodeficiency virus envelope protein and the second epitope is from a second human immunodeficiency virus envelope protein.

14. The recombinant immunoglobulin M composition of claim 12, wherein the first epitope is an epitope of a human immunodeficiency virus envelope protein gp120.

15. The recombinant immunoglobulin M composition of claim 12, wherein the first epitope is a carbohydrate dependent epitope related to the V3 loop of the human immunodeficiency virus envelope protein gp120.

16. The recombinant immunoglobulin M composition of claim 12, wherein the second epitope is a part of a V2 loop of the human immunodeficiency virus envelope protein gp120.

17. The recombinant immunoglobulin M composition of claim 12, wherein the second epitope is a part of a CD4 binding site of the human immunodeficiency virus envelope protein gp120.

18. The recombinant immunoglobulin M composition of claim 11, wherein the variable region of the first light chain is connected to the variable region of the first heavy chain, which is connected to the first constant region.

19. The recombinant immunoglobulin M composition of claim 11, wherein the variable region of the second light chain is connected to the variable region of the second heavy chain, which is connected to the second constant region.

20. The recombinant immunoglobulin M composition of claim 11, wherein the first constant region is linked to the first variable region of the first heavy chain via a first glycine- and serine-rich linker and the second constant region is linked to the second variable region of the second heavy chain via a second glycine- and serine-rich linker.

21. The recombinant immunoglobulin M composition of claim 11, further comprising a joining chain connecting the first constant region of a heavy chain of a first monomer to a third constant region of a heavy chain of a second adjacent monomer to form a pentameric immunoglobulin M composition.
